(19) United States
(12) Patent Application Publication
(10) Pub. No.: US 2006/0095005 A1
(43) Pub. Date: May 4, 2006

(54) Multi-exchange catheter guide member with integral seal

(75) Inventors: David Quinn, Salthill (IE); Kevin Boyle, Renmore (IE); Niall Duffy, Tuam (IE); Gerry Clarke, Galway (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/980,555

(22) Filed: Nov. 3, 2004

(51) Int. Cl. A61M 29/00

(52) U.S. Cl. 604/103.04; 606/108; 606/192

(57) ABSTRACT

A catheter and a guidewire exchange system includes a catheter and a guide member. The catheter includes a lumen extending through the shaft and sized to receive the guidewire, and a longitudinal guideway enabling transverse access from the shaft exterior surface to the lumen. The guide member includes a housing, a catheter passageway extending through the housing and adapted to slidably receive the catheter, a guidewire passageway extending from one end of the housing into the catheter passageway and including a tube adapted to merge the guidewire transversely through the guideway and into the first lumen, and an fluid flow reduction body that is positioned in the guidewire passageway and impedes fluid flow therethrough.

### TECHNICAL FIELD

The present invention generally relates to medical catheters and medical apparatuses involving medical catheters. The present invention more particularly relates to mechanical devices adapted to improve the seal between the catheter and hemostasis valve during use.

### BACKGROUND

Cardiovascular disease, including atherosclerosis, is a leading cause of death in the U.S. The medical community has developed a number of methods and devices for treating coronary heart disease, some of which are specifically designed to treat the complications resulting from atherosclerosis and other forms of coronary arterial narrowing.

One method for treating atherosclerosis and other forms of coronary narrowing is percutaneous transluminal coronary angioplasty, commonly referred to as “angioplasty” or “PTCA.” The objective in angioplasty is to enlarge the lumen of the affected coronary artery by radial hydraulic expansion. The procedure is accomplished by inflating a balloon of a balloon catheter within the narrowed lumen of coronary artery.

In addition to PTCA, catheters are used for delivery of stents or grafts, therapeutic drugs (such as anti-vaso-occlusion agents or tumor treatment drugs) and radiopaque agents for radiographic viewing. Other uses for such catheters are well known in the art.

The anatomy of coronary arteries varies widely from patient to patient. Often a patient's coronary arteries are irregularly shaped, highly tortuous and very narrow. The tortuous configuration of the arteries may present difficulties to the physician in proper placement of a guidewire, and advancement of a catheter to a treatment site. A highly tortuous coronary anatomy typically will present considerable resistance to advancement of the catheter over the guidewire.

Therefore, it is important for a catheter to be highly flexible. However, it is also important for a catheter shaft to be stiff enough to push the catheter into the vessel in a controlled manner from a position far away from the distalmost point of the catheter.

Catheters for PTCA and other procedures may include a proximal shaft, a transition section and a distal shaft having a flexible distal tip. In particular, the catheters have a proximal shaft, which is generally rigid for increased pushability and a more flexible distal shaft with a flexible distal tip for curving around particularly tortuous vessels. The proximal shaft may be made stiff by the insertion of a thin biocompatible tube, such as a stainless steel hypotube, into a lumen formed within the proximal shaft. The transition section is the portion of the catheter between the stiffer proximal shaft and the more flexible distal shaft, which provides a transition in flexibility between the two portions.

With some types of catheter construction, when an increase in resistance occurs during a procedure there is a tendency for portions of the catheter to collapse, buckle axially or kink, particularly in an area where flexibility of the catheter shaft shifts dramatically. Consequently, the transition section is often an area where the flexibility of the catheter gradually transitions between the stiff proximal shaft and the flexible distal shaft. It is known in the art to create a more gradual flexibility transition by spiral cutting a distal end of the hypotubing used to create stiffness in the proximal shaft. Typically, the spiral cut is longitudinally spaced father apart at the hypotube proximal end creating an area of flexibility, and longitudinally spaced closer together at the hypotube distal end creating an area of even greater flexibility.

In a typical PTCA procedure, it may be necessary to perform multiple dilatations, for example, using various sized balloons. In order to accomplish the multiple dilatations, the original catheter must be removed and a second catheter tracked to the treatment site. When catheter exchange is desired, it is advantageous to leave the guidewire in place while the first catheter is removed to properly track the second catheter.

Two types of catheters commonly used in angioplasty procedures are referred to as over-the-wire (OTW) catheters and rapid exchange (RX) catheters. A third type of catheter with preferred features of both OTW and RX catheters, which is sold under the trademarks MULTI-EXCHANGE, ZIPPER MX, ZIPPER, MX and/or MXII, is discussed below. An OTW catheter's guidewire lumen runs the entire length of the catheter and may be positioned next to, or enveloped within, an inflation shaft. Thus, the entire length of an OTW catheter is tracked over a guidewire during a PTCA procedure. A RX catheter, on the other hand, has a guidewire lumen that extends within only the distalmost portion of the catheter. Thus, during a PTCA procedure only the distalmost portion of a RX catheter is tracked over a guidewire.

If a catheter exchange is required while using a standard OTW catheter, the user must add an extension wire onto the proximal end of the guidewire to maintain control of the guidewire, slide the catheter off of the extended guidewire, slide the new catheter onto the guidewire and track back into position. Multiple operators are required to hold the extended guidewire in place while the original catheter is exchanged in order to maintain its sterility.

A RX catheter avoids the need for multiple operators when exchanging the catheter. With a rapid exchange catheter, the guidewire runs along the exterior of the catheter for all but the distalmost portion of the catheter. As such, the guidewire can be held in place without an extension when the catheter is removed from the body. However, one problem associated with RX catheters is the guidewire, and most of the catheter, must be removed from the body in order to exchange guidewires. Essentially the procedure must then start anew because both the guidewire and the catheter must be retracked to the treatment site. An OTW catheter, with the guidewire lumen extending the entire length of the catheter, allows for simple guidewire exchange.

A balloon catheter capable of both fast and simple guidewire and catheter exchange is particularly advantageous. A catheter designed to address this need is sold by Medtronic Vascular, Inc. of Santa Rosa, Calif. under the trademarks MULTI-EXCHANGE, ZIPPER MX, ZIPPER, MX and/or MXII (hereinafter referred to as the “MX catheter”). An MX catheter is disclosed in U.S. Pat. No. 4,988,356 to Crittenden et al.; co-pending U.S. patent application Ser. No. 10/116,234, filed Apr. 4, 2002; co-pending U.S. patent application Ser. No. 10/251,578, filed Sep. 18, 2002; co-pending U.S. patent application Ser. No. 10/251,477, filed Sep. 20, 2002; co-pending U.S. patent application Ser. No. 10/722,191, filed Nov. 24, 2003; and co-pending U.S. patent application Ser. No. 10/720,535, filed Nov. 24, 2003, all of which are incorporated by reference in their entirety herein.

The MX catheter includes a catheter shaft having a guidewire lumen positioned side-by-side with an inflation lumen. The MX catheter also includes a longitudinal cut that extends along the catheter shaft and that extends radially from the guidewire lumen to an exterior surface of a catheter shaft. A guide member through which the shaft is slidably coupled cooperates with the longitudinal cut such that a guidewire may extend transversely into or out of the guidewire lumen at any location along the longitudinal cut's length. By moving the shaft with respect to the guide member, the effective over-the-wire length of the MX catheter is adjustable.

The guidewire is threaded into a guidewire lumen opening at the distal end of the catheter and out through the guide member. The guidewire lumen envelopes the guidewire as the catheter is advanced into the patient's vasculature while the guide member and guidewire are held stationary. Furthermore, the indwelling catheter may be removed by withdrawing the catheter from the patient while holding the proximal end of the guidewire and the guide member in a fixed position. When the catheter has been withdrawn to the point where the distal end of the cut has reached the guide member, the distal portion of the catheter over the guidewire is of a sufficiently short length that the catheter may be drawn over the proximal end of the guidewire without releasing control of the guidewire or disturbing its position within the patient.

While MX catheters provide many advantages over RX and OTW catheters, both RX and MX catheters need to be sealed effectively at the hemostasis valve. OTW catheters are readily sealed at the valve since the guidewire is within the catheter shaft which extends through the valve. RX and MX catheters have a catheter shaft and guidewire separated proximal to the hemostasis valve and thus an effective valve seal must take into consideration the catheter and guidewire separation for an RX catheter and with the guide member in the case of the MX catheter. For example, in a typical dye injection, the physician may pull a slight negative pressure to ensure no air bubbles are within the system prior to injecting the dye. If the physician pulls a very heavy negative pressure, there remains a possibility that air may enter the patient through the hemostasis valve if not sealed sufficiently around the catheter, guide wire and guide member of an MX catheter. Similarly, when a hemostasis valve has an active/passive gasket, if the valve is not properly closed down on an RX catheter shaft and guidewire, air may be drawn into the system when a very heavy vacuum is drawn.

Accordingly, it is desirable to provide an apparatus that can reduce or eliminate the opportunity for unwanted air aspiration at the hemostasis valve when using a MX catheter. In addition, it is desirable to provide such an apparatus that does not slow down guidewire insertion or other medical processes involving the catheter. Furthermore, other desirable features and characteristics of the present invention will become apparent from the subsequent detailed description and the appended claims, taken in conjunction with the accompanying drawings and the foregoing technical field and background.

### BRIEF SUMMARY

A system is provided for exchanging a catheter and a guidewire. The system includes a catheter with a guide member. The catheter includes an elongate shaft having an exterior surface, a proximal end, and a distal end; a first lumen extending through the shaft from the shaft proximal end to the shaft distal end, and sized to receive a guidewire; and a longitudinal guideway extending distally from the shaft proximal end, and enabling transverse access from the shaft exterior surface to the first lumen. The guide member includes a housing having a proximal end and a distal end; a catheter passageway extending through the housing from the proximal end to the distal end and adapted to slidably receive the catheter; a guidewire passageway extending from the housing proximal end into the catheter passageway, and including a tube adapted to merge the guidewire transversely through the guideway and into the first lumen; and sealing body that is positioned in the guidewire passageway.

A guide apparatus is also provided for advancing and retracting a guidewire and a catheter having a lumen, an exterior surface, and a longitudinal guideway that enables transverse access from the catheter exterior surface to the lumen in a patient. The apparatus includes a housing having a proximal end and a distal end; a catheter passageway extending through the housing from the proximal end to the distal end and adapted to slidably receive the catheter; a guidewire passageway extending from the housing proximal end into the catheter passageway, and comprising a tube adapted to merge the guidewire transversely through the guideway and into the first lumen; and sealing body that is positioned in the guidewire passageway.

### BRIEF DESCRIPTION OF THE DRAWINGS

The present invention will hereinafter be described in conjunction with the following drawing figures, wherein like numerals denote like elements, and

FIG. 1 is a perspective view of a guide member with a guide wire extending through a guide member and into a catheter according to the present invention;

FIGS. 2A-D are cross sectional views of a catheter at points A-A, B-B, C—C, and D-D illustrated in FIG. 1;

FIG. 3 is a perspective cross sectional view of an oval proximal shaft;

FIG. 4 is a cross sectional view of a circular proximal shaft;

FIG. 5 is a sectional view of a guide member and its components according to the present invention;

FIG. 6 is a perspective view of a guide member that is sectioned to depict a guidewire tube extending through a passageway within the guide member according to an embodiment of a passive seal according to the present invention;

FIG. 7 is a sectional view of an o-ring body as another embodiment of a passive seal positioned in the guidewire entrance port according to the present invention;

FIG. 8 is a sectional view of a labyrinth type seal as another embodiment of a passive seal positioned in the guidewire entrance port according to the present invention; and

FIG. 9 is a sectional view of a gel seal as another embodiment of a passive seal positioned in the guidewire entrance port according to the present invention;

FIG. 10 is a sectional view of a quad ring seal as an embodiment of an active seal positioned in the guidewire entrance port according to the present invention;

FIG. 11 is a sectional view of a half quad ring seal as another embodiment of an active seal positioned in the guidewire entrance port according to the present invention;

FIG. 12 is a sectional view of an hour glass seal as another embodiment of an active seal positioned in the guidewire entrance port according to the present invention;

FIG. 13 is a sectional view of a rocking seal as another embodiment of an active seal positioned in the guidewire entrance port according to the present invention;

FIG. 14 is a sectional view of a half rocking seal as another embodiment of an active seal positioned in the guidewire entrance port according to the present invention;

FIGS. 15A and 15B are sectional views of a guide member with a passageway that is modified to include flexible flaps according to an embodiment of the present invention; and

FIGS. 16A and 16B are sectional views of a guide member with a passageway that is modified to include an inflatable balloon according to an embodiment of the present invention.

### DETAILED DESCRIPTION

The following detailed description is merely exemplary in nature and is not intended to limit the invention or the application and uses of the invention. Furthermore, there is no intention to be bound by any expressed or implied theory presented in the preceding technical field, background, brief summary or the following detailed description.

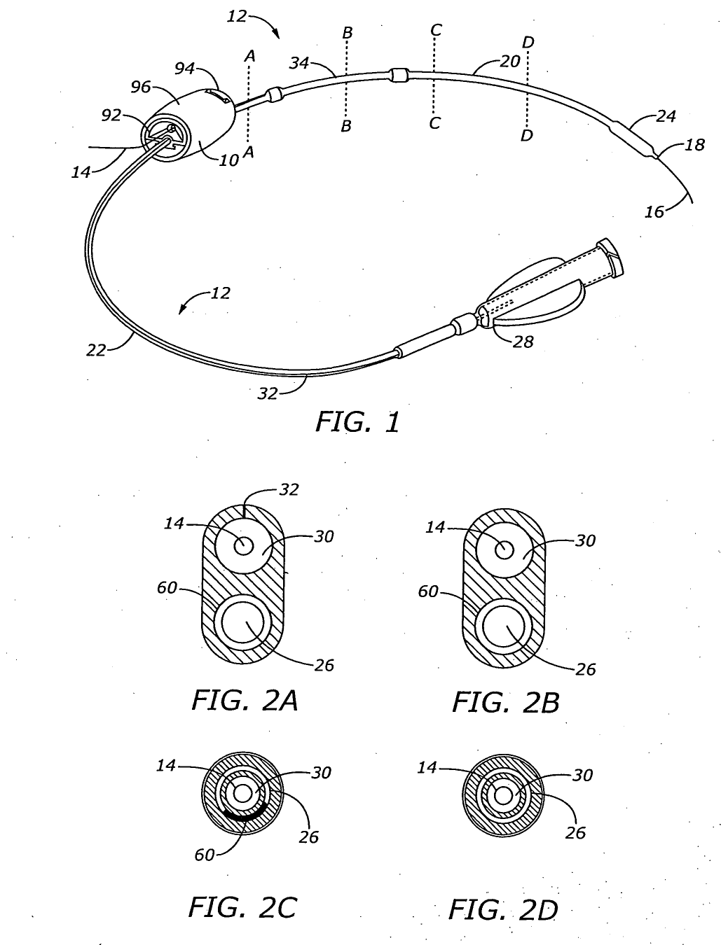

The present invention is used with an MX catheter, an exemplary embodiment of which is illustrated in FIG. 1. The catheter 12 includes an elongate, flexible, cylindrical main body having a distal shaft 20 and a proximal shaft 22. According to the present embodiment, the catheter 12 is a delivery catheter for such procedures as PTCA or stent delivery and has a balloon 24 mounted around the catheter body near the catheter distal end 18. The balloon 24 may be inflated and deflated through the catheter inflation lumen 26. The inflation lumen 26 communicates with a fitting 28 at the catheter proximal end, and extends the catheter length to terminate in communication with the balloon interior at the catheter distal end 18. The catheter 12 also includes a guidewire lumen 30 that receives the guidewire 14 and extends the entire catheter length. A longitudinal cut extends into the guidewire lumen 30 along the length of most of the proximal shaft 22 to form a guideway 32. The proximal shaft distal section 34 does not include the guideway 32. The guidewire lumen 30 and the inflation lumen 26 are coaxially arranged in the distal shaft 20 according to the present embodiment.

The present invention includes a guide member for the MX catheter 12. FIG. 1 depicts a guide member 10 according to an embodiment of the invention, with a guide wire 14 extending through the guide member 10 and into the MX catheter 12. FIGS. 2A to 2D are cross sections of the catheter 12 at points A-A, B-B, C—C, and D-D along the catheter length. The guide member 10 serves as a juncture in which the catheter 12 and guidewire 14 may be merged or separated so that the guidewire portion that extends proximal to the guide member 10 is separated from the catheter 12, and the guidewire portion that is located distal to the guide member 10 is contained and housed within the catheter, although the guidewire distal end 16 may protrude out of the catheter distal end 18.

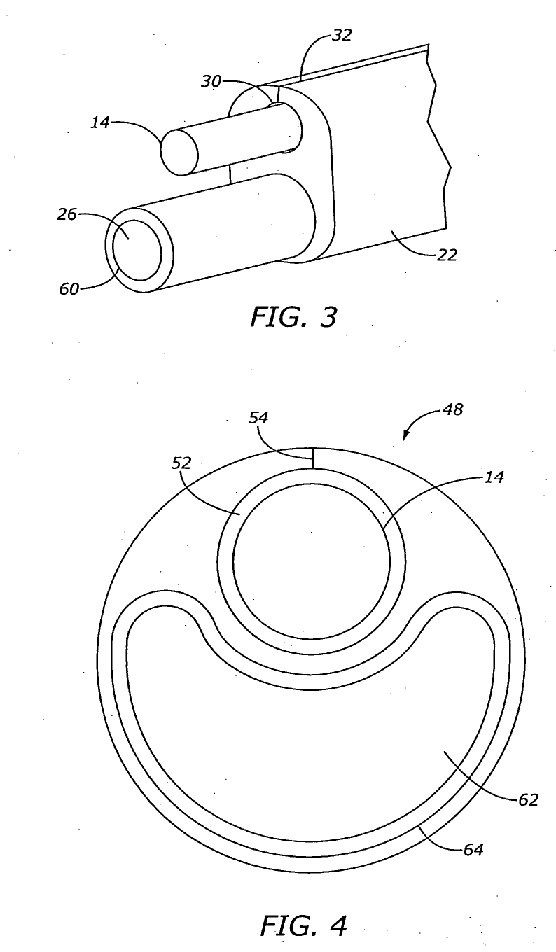

The catheter proximal shaft 22 described above can be modified to suit various needs. For example, the proximal shaft can be a tri-lumen shaft to provide passage for various drugs, fluids, wires, or other necessary compositions or equipment. Further, the proximal shaft may be oval, circular, or other suitable shape. FIG. 3 is a perspective cross sectional view of an oval proximal shaft 22 according to one embodiment of the invention, and FIG. 4 is a cross sectional view of a circular proximal shaft 48 according to another embodiment of the invention. Each of the proximal shafts 22, 48 has a respective guidewire lumen 30, 52 that is accessible through a guideway 32, 54 located along the proximal shaft length. Each of the proximal shafts 22, 48 also includes an inflation lumen 26, 62 that extends side by side with the guidewire lumen 30, 52 along the proximal shaft length. The inflation lumens 26, 62 are preferably supported by a stiffening member 60, 64 such as a hypotube. The inflation lumen 62 in the embodiment depicted in FIG. 4 is crescent shaped and the hypotube stiffening member 64 also is formed in the same shape to withstand force transmission along the catheter length. The stiffening members may further include a transition section at their respective distal sections in conjunction with a transition between the relatively stiff proximal shaft to the relatively flexible distal shaft and avoid shaft kinking at the junction therebetween. For example, the hypotube 60 may be skived at its distal end, with the skived portion extending into the distal section as depicted in FIG. 2C.

Returning to FIG. 1, the proximal shaft 22 can be formed from suitable biomedical grade materials such as polyethylene, cross-linked polyethylene, polyolefins, polyamides, blends of polyamides and polyolefins, fluoropolymers, polyesters, polyketones, polyimides, polysulphones, polyoxymethylenes, and compatibilizers based on polyolefins, including grafted polyolefins, and other comparable materials. A lubrication additive may also be used with any polymer and may include polyethylene micro-powders, fluoropolymers, silicone based oils, fluoro-ether oils, molybdenum disulphide and polyethylene oxide. Additionally, a reinforcing additive may be used such as nano-clays, graphite, carbon fibers, glass fibers, and polymeric fibers. The distal shaft 20 can be made of a suitable polyethylene or polyolefin that readily bonds to the proximal shaft 22.

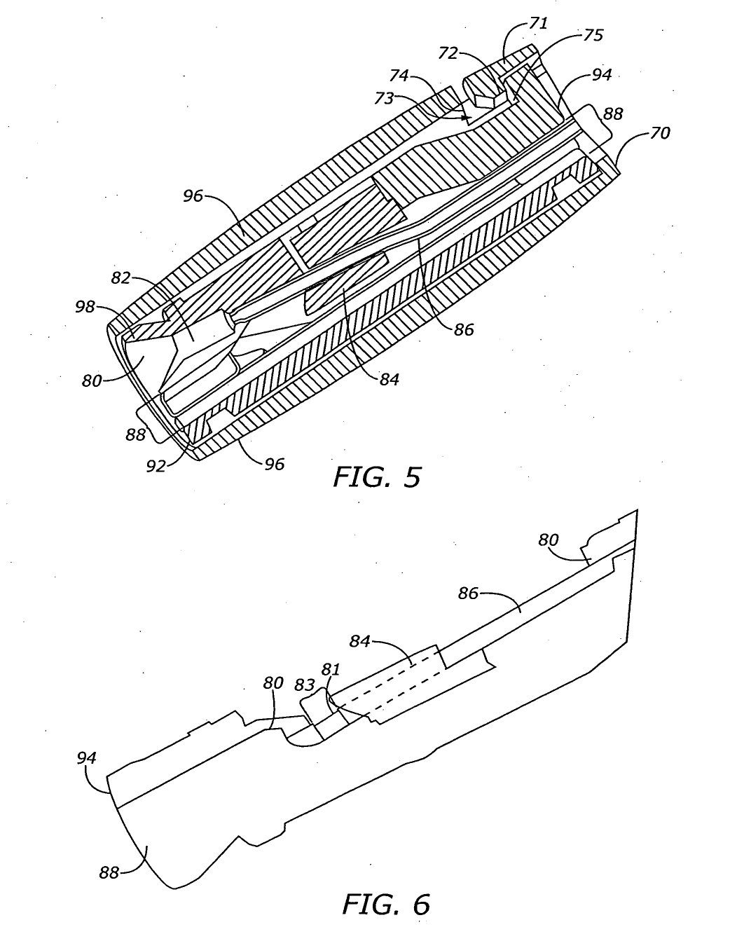

Turning now to FIG. 5, the guide member 10 and its components will be discussed according to one embodiment of the invention. The guide member 10 surrounds the proximal shaft 22 and includes proximal and distal ends 92, 94. An outer tubular member 96 freely rotates around an inner main body 98 and hence is decoupled from the inner main body 98. An inwardly extending distal annular wall 70 prevents the main body 98 from slipping out of the outer member 96. A retaining clip 71 includes a tab 72 that extends into a space 73 formed by two main body walls 74, 75. Additional tabs may be used as necessary to retain the inner main body 98 within the tubular member 96.

The guide member main body 98 includes a catheter passageway 88 extending longitudinally in a generally straight line from the guide member proximal end 92 to the guide member distal end 94. A guidewire passageway 80 extends distally from the guide member proximal end 92 through an entrance port 82 into a tube 86 and then into the catheter guidewire lumen 30, although the catheter is not depicted in FIG. 5. The passageway 80 is configured to slidingly receive the proximal shaft 22, and has a cross sectional shape that approximates the proximal shaft shape, whether the proximal shaft is circular, oval, triangular, shamrock shaped, or otherwise shaped. The passageway 80 enlarges in a central area to provide space for a keel 84 that is aligned with the passageway 80 and positioned to spread the catheter guideway 32 and extend into the catheter guidewire lumen 30 to enable guidewire insertion during use.

The entrance port 82 is configured to mate with a conventional wire introducer tool and is tapered to aid in loading such a tool. The tube 86 may vary in its length, although in an exemplary embodiment of the invention the tube 86 extends through the catheter guidewire lumen 30 approximately thirty-five millimeters past the guide member distal end 94. The tube 86 may be formed from a flexible material such as a polyimide, and particularly the tube region that extends through the catheter guidewire lumen 30. In one embodiment of the invention the tube region that introduces the guidewire 14 into the guidewire lumen 30 may be substantially rigid to provide the necessary support for the guidewire 14.

The guide member 10 is made of blends of polyamides and polyolefins in an exemplary embodiment of the invention. Other exemplary materials include ceramics, metals such as stainless steel, and other polymers such as polyamides and liquid crystal polymers. Lubrication additives such as polyethylene micro-powders, fluoropolymers, silicone-based oils, fluoro-ether oils, molybdenum disulphide, and polyethylene oxide may be included. Reinforcing additives such as nano-clays, graphite, carbon fibers, glass fibers, polyesters, polyketones, polyimides, polysulphones, polyoxymethylenes, polyolefins, cross-linked polyolefins may also be included, along with compatibilizers based on polyolefins, such as grafted polyolefins, ceramics, and metals.

An exemplary guide member operation will now be described, although the procedures in the following description clearly set forth only one of many operations enabled by the guide member 10. After the guidewire 14 and a guide catheter (not shown) are inserted into a patient, the catheter 12 is inserted with a backloading operation. The guidewire 14 is inserted into the catheter distal end 18 and threaded proximally through the guidewire lumen 30 until the guidewire tube 86 captures the guidewire proximal end and directs it into the passageway 80 and then out of the guide member proximal end 92. This procedure can be accomplished with the guide member 10 adjacent the catheter guideway distal end. As the distal shaft 20 enters the patient, the guide member 10 will reach the hemostatic valve (not shown). The guide member 10 is not intended to enter the valve and is seated adjacent to the valve. The proximal shaft 22 is then advanced through the guide member, and the keel 84 engages the catheter guideway 32. After the catheter 12 is inserted, the hemostatic valve may be closed down on the catheter shaft at a region that is distal to the guide member 10. Since the tube 86 extends in to the distal shaft 20, it is subjected to the valve clamping force. If a wire change is required, one simply withdraws the guidewire 14 from the guide member 10 as the guide member 10 is seated against the valve and as the proximal shaft 22 remains in the patient. A new guidewire is then inserted into the catheter through the passageway 80. If a catheter exchange is required, one simply holds the guidewire 14 in place and begins moving the proximal shaft 22 proximally through the guide member. Another catheter may then be backloaded onto the guidewire 14 and introduced into the patient as described above after the catheter has been separated from the guidewire.

Passageway 80 is adapted to include a seal that prevents or minimizes fluid movement through the passageway. Exemplary airflow reduction bodies according to the present invention are described below and categorized as either passive seals or active seals. Passive seals are generally defined as bodies that prevent or substantially minimize fluidflow without changing shape or orientation. Active seals are generally defined as bodies that minimize fluid flow without changing shape or orientation, but may substantially minimize or entirely prevent fluid flow due to a change of shape or orientation. Such a change of shape is typically caused due to the force of inrushing fluid on one side of the airflow reduction body when the guidewire 14 is advanced through the passageway 80.

The following four seals are all exemplary passive seals. FIG. 6 is a perspective view of the guide member 10 with a modified passageway according to one embodiment of a passive seal. The guide member 10 is sectioned in the figure in order to depict the guidewire tube 86 extending through the passageway 80. The tube 86 has a substantially uniform inner diameter, but includes a fixed reduced diameter region 83 that prevents air movement therethrough. The term “fixed” in this sense means that the reduced diameter region 83 is a passive, unchanging airflow reduction body unlike some of the active airflow reduction bodies described hereinafter. The small diameter region 83 has a smaller diameter than the rest of the tube 86, or at least a diameter that is smaller than the small diameter region's immediate or nearby vicinity, and consequently substantially reduces the amount of air that flows through the tube 86 without impeding guidewire movement. The small diameter region 83 is formed proximally with respect to the keel 84, as depicted in FIG. 6. However, the small diameter region 83 may be formed elsewhere within the passageway, or even distal to the guide member 10 if the tube 86 extends beyond the guide member within the guidewire lumen 30.

The small diameter region 83 may be formed by slightly constricting the tube 86 using an annular body 81 that is connected to the tube. In an exemplary embodiment of the invention, the annular body 81 is a bracket, a clamp, a sleeve, or other device. The annular body 81 may surround the tube outer surface. Alternatively, the annular body 81 may also be attached to the tube interior surface. In another exemplary embodiment, the bracket 81 interrupts the continuity of the polyimide or other tube material, and is manufactured in-line with the tube 86. In such an embodiment, the tube 86 is joined to the bracket 81 by applying heat, an adhesive, or any other suitable joining tool or composition.

In another exemplary embodiment, an annular body is not used to create the small diameter region 83. Rather, the polyimide or other tube material is simply manufactured to have a discrete region that has a smaller diameter than the rest of the tube 86, or at least a smaller diameter than that of the discrete region's immediate or nearby vicinity.

One reason that the small diameter region 83 is highly effective at restricting fluid passage through the tube 86 is the seal uniformity across the region 83. A full seal entirely surrounding tube 86 is preferred. Fluid flow prevention also is found to be positively related to the length of the longitudinal length of the small diameter region 83. Consequently, doubling the small diameter region length has the effect of approximately doubling the resistance to fluid flow.

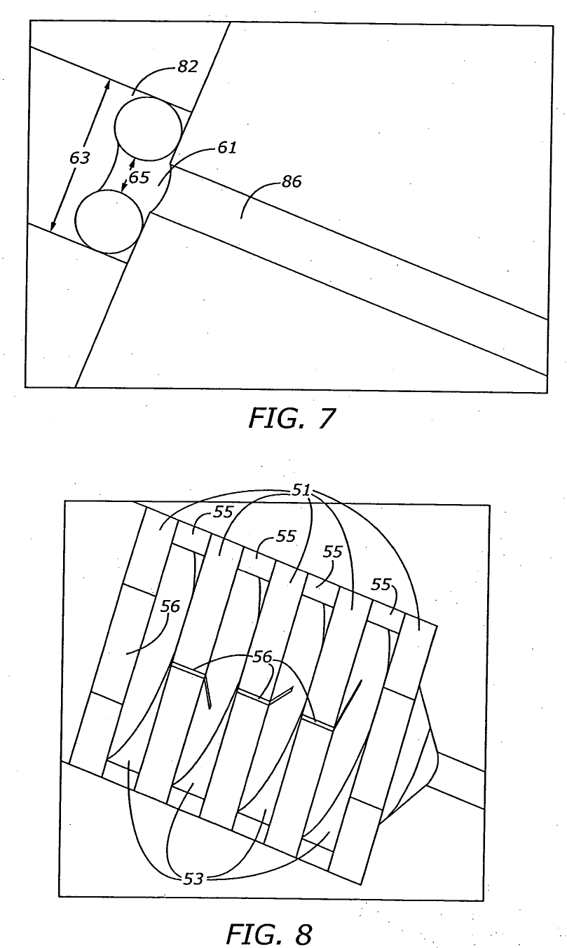

FIG. 7 is a sectional view of an o-ring body 61 as another embodiment of a passive seal, positioned in the guidewire entrance port 82 adjacent to the tube 86. The o-ring body can be formed of a flexible material, although a substantially rigid material will reduce friction with the guidewire 14. The o-ring body 61 can be positioned in any suitable location in the guidewire passageway 80 to effectively prevent or substantially minimize fluid flow therethrough. An exemplary location for the o-ring body 61 is the entrance port 82, although the o-ring body 61 may be in any suitable position within the guidewire passageway 80, including the tube 86 or even the keel 84. The o-ring body 61 has an outer diameter 63 that can approximate the inner diameter of the guidewire passageway area in which the o-ring body is positioned. The o-ring body 61 also has an inner diameter 65 that approximates the guidewire diameter in order to provide a substantially fluid tight seal with the guidewire 14.

FIG. 8 is a sectional view of a labyrinth type seal as another embodiment of a passive seal, positioned in the guidewire entrance port 82 adjacent to the tube 86. The labyrinth seal can be positioned in any suitable location in the guidewire passageway 80 to effectively prevent or substantially minimize fluid flow therethrough. An exemplary location for the labyrinth seal is the entrance port 82, although the seal may be in any suitable position within the guidewire passageway 80.

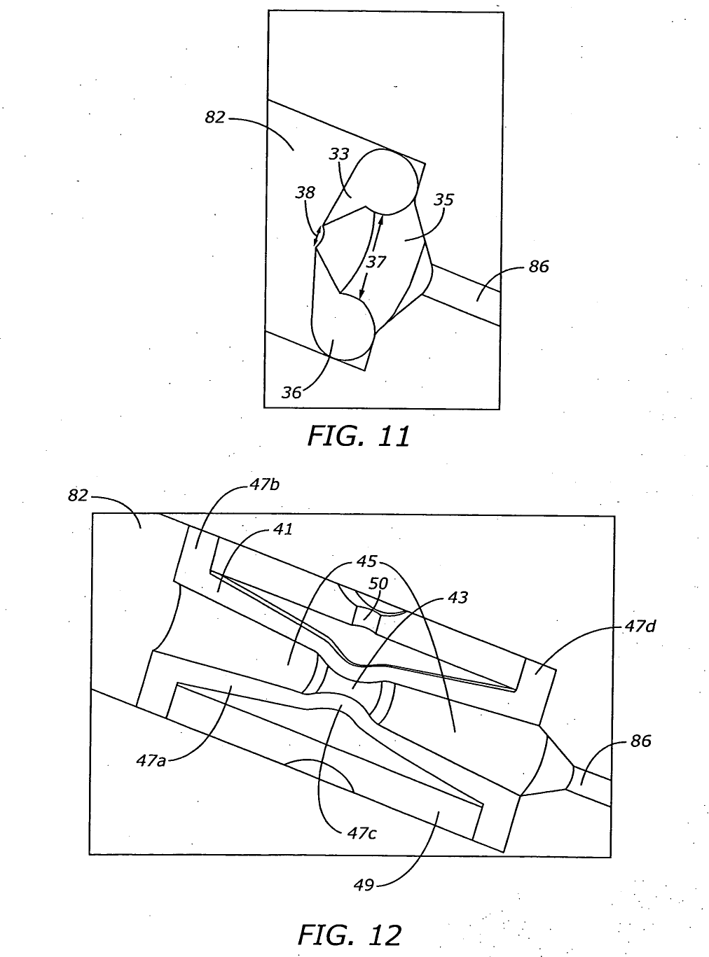

The labyrinth seal includes a set of stacked disks 51 with an air space 53 between each pair of disks 51. Each disk 51 includes a slit 56 wide enough to slidably receive the guidewire 14. Each slit 56 is shorter than the disk diameter and is provided to allow the flow of only a small amount of inrushing fluid. In an exemplary embodiment, each slit 56 is rotated with respect to a slit 56 in an adjacent disk 51 to prevent a direct pattern for fluid flow. For example, the slit in each disk in the embodiment depicted in FIG. 8 is oriented at a 45° angle with respect to the slit in the adjacent disk. Consequently, the force produced by inrushing fluid caused by factors such as a pressure drop due to vacuum drawn on guide catheter, or by guidewire advancement, is substantially eliminated after one or more disks have blocked and distributed the force. Each disk 51 is separated by spacing bodies 55 that are o-shaped bodies in an exemplary embodiment. The disks 51 are separated by a width that is close to the disk width in the embodiment depicted in FIG. 11, although the spacing can be adjusted to optimize the distribution of inrushing fluid and buffer the force that the inrushing fluid creates on the proximal side of each disk 51. Additional disks can be added, or fewer disks can be utilized in order to effectively block air passage, and as few as one disk can be utilized in some cases.

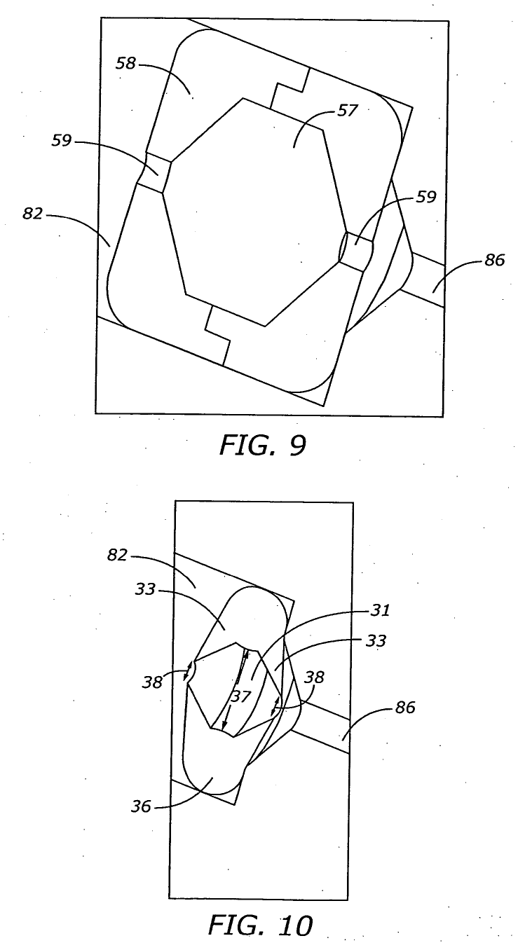

FIG. 9 is a sectional view of a gel seal as another embodiment of a passive seal, positioned in the guidewire entrance port 81 adjacent to the tube 86. The gel seal can be positioned in any suitable location in the guidewire passageway 80 to effectively prevent or substantially minimize fluid flow therethrough. An exemplary location for the gel seal is the entrance port 82, although the seal may be in any suitable position within the guidewire passageway 80.

The gel seal includes a gel center 57 that is secured in the guidewire passageway 80 using a capsule 58. The gel center 57 may include any biocompatible gel composition such as silicone or another polymer suspended in a biocompatible dispersion medium such as water or a saline solution. The polymer is viscous enough to prevent leakage through guidewire access holes 59 molded in the capsule 58. The polymer is also sufficiently fluid to allow the guidewire 14 to advance or retract with little to negligible friction. The capsule 14 is a molded body formed from two separate pieces in the exemplary embodiment depicted in FIG. 9, although the capsule 14 can be any housing that essentially encapsulates the gel center 57 to prevent the gel from leaking when the guide member 10 is inverted or rotated.

The following seven seals are all exemplary active seals. FIG. 10 is a sectional view of a quad ring active seal 31 positioned in the guidewire entrance port 81 adjacent to the tube 86, and FIG. 11 is a sectional view of a half quad ring active seal 35 similarly positioned. The quad ring seal 13 or the half quad ring seal 35 can be positioned in any suitable location in the guidewire passageway 80 to effectively prevent or substantially minimize fluid flow therethrough. An exemplary location for either of these seals is the entrance port 82, although the seals may be secured in any suitable position within the guidewire passageway 80.

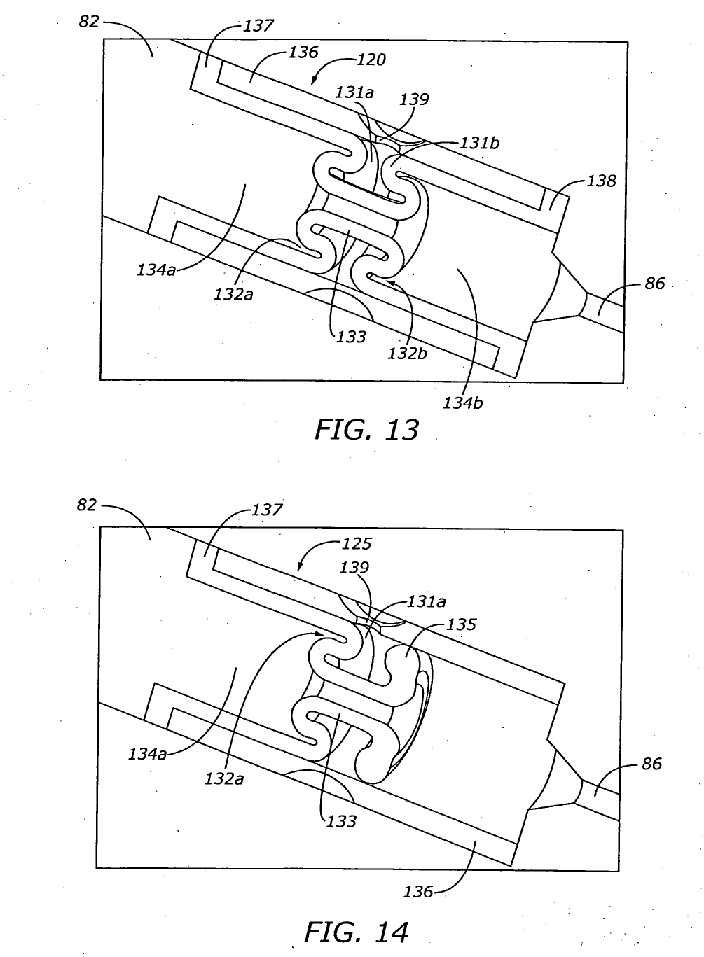

Both the quad ring seal 31 and half quad ring seal 35 include an annular central member 36 that includes an inner diameter 37 that is slightly larger than the guidewire diameter. The central member 36 is essentially an o-ring body. In the half quad ring seal 35, the central member 36 is formed continuous with an annular flexible body 33 that is foldable against the guidewire 14 to prevent inrushing fluid from reaching the catheter guidewire lumen 30. The flexible body 33 has a central aperture 38 for slidingly receiving the guidewire 14. The central aperture 38 has a diameter that is slightly larger than the guidewire diameter, but is smaller than the central member inner diameter 37. The flexible body 33 is formed as a cone with a truncated tip that produces the central aperture 38. The flexible body 33 is configured to collapse from the conical shape to form a fluid tight seal around the guidewire 14 as a result of the force from inrushing fluid. As shown in FIG. 13, the quad ring seal 31 includes two flexible bodies 33 that flank the central member 36. The two flexible bodies 33 are disposed oppositely with respect to the central member 36.

FIG. 12 is a sectional view of an hour glass active seal 41 positioned in the guidewire entrance port 81 adjacent to the tube 86. The hour glass seal 41 can be positioned in any suitable location in the guidewire passageway 80 to effectively prevent or substantially minimize fluid flow therethrough. An exemplary location for these seal 41 is the entrance port 82, although the seal 41 may be secured in any suitable position within the guidewire passageway 80.

The hour glass seal 41 includes a continuous molded body 47a-d that is hollowed to define a passageway 45 through which the guidewire 14 extends. The passageway 45 narrows as it extends from the seal proximal end 47b to a small diameter neck 43. From the neck 43, the passageway 45 widens until it reaches the seal distal end 47d. In an exemplary embodiment, the passageway 45 has a symmetrical configuration, meaning that the passageway 45 on the proximal side of the neck 43 is identical to the passageway 45 on the distal side of the neck 43. The neck 43 has a smaller diameter than the rest of the passageway 45, and the neck diameter is sized to slidably receive the guidewire 14. The neck diameter is also slightly larger than the guidewire diameter, and the neck 43 is defined by a hollow flexible wall 47c that is continuously formed with the remaining molded body 47a, b, d. The force of inrushing fluid against the walls 47a causes the neck 43 to constrict as the flexible material 47c changes shape to form a substantially fluid tight seal around the guidewire 14. To ensure that the wall 47c changes shape and thereby causes the neck 43 to constrict due to the force of inrushing fluid, the wall 47c is substantially less rigid than the adjacent walls 47a in an exemplary embodiment of the invention. In order to make the wall 47c substantially less rigid wall than the adjacent walls 47a, the wall 47c can be substantially thinner than the adjacent walls 47a, or the wall 47 can be formed from a less rigid material than that of the adjacent walls 47a.

If the walls 47a in the molded body are not rigid enough, or do not span a sufficient width, to support the entire hour glass seal 41 together with the walls that define the guidewire passageway 80, the walls can be radially extended at the seal proximal and distal ends 47b, 47d as depicted in FIG. 12. Further, a rigid support member 49 can be provided around a periphery of the hour glass seal 41 to provide further support if necessary. If the rigid support member 49 conforms or nearly conforms to the hour glass seal periphery, a vent 50 may be included in the support member 49. The vent 50 allows external fluid to fill any space between the support member 49 and the hour glass seal 41, and consequently enables the wall 47c to constrict the neck 43.

FIG. 13 is a sectional view of a rocking seal 120 positioned in the guidewire entrance port 81 adjacent to the tube 86. FIG. 14 is a sectional view of a half rocking seal 125 similarly positioned. The rocking seal 120 or half rocking seal 125 can be positioned in any suitable location in the guidewire passageway 80 to effectively prevent or substantially minimize airflow therethrough. An exemplary location for these seals 120, 125 is the entrance port 82, although the seals 120, 125 may be secured in any suitable position within the guidewire passageway 80.

The rocking seal 120 is a continuously formed structure that includes two substantially cylindrical walls 134a, 134b that are continuously joined with an elongate neck 133. The elongate neck 133 is defined by a wall that is formed of a flexible material and has a diameter that is smaller than the diameter formed by the cylindrical walls 134a, 134b. The elongate neck inner diameter is slightly larger than the guidewire diameter, and is sized to slidingly receive the guidewire 14. The cylindrical walls 134a, 134b and the elongate neck 133 are joined by folded walls 131a, 131b that double back inwardly and form annular pockets 132a, 132b that surround a portion of the elongate neck 133.

When the guidewire passageway 80 is subjected to a vacuum force, inrushing fluid is received by the annular pocket 132a that is proximal to the elongate neck 133. The force of the inrushing air pushes the folded wall 131a toward folded wall 131b. The wall that defines the elongate neck 133 is flexible and consequently buckles or otherwise changes shape, causing the elongate neck inner diameter to constrict so that a substantially airtight seal is formed around the guidewire 14. To ensure that the wall defining the elongate neck 133 changes shape and thereby causes the elongate neck 133 to constrict due to the force of inrushing fluid, the wall is substantially less rigid than the adjacent folded walls 131a, 131b and the cylindrical walls 134a, 134b in an exemplary embodiment of the invention. In order to make the wall defining the elongate neck 133 substantially less rigid, the wall can be substantially thinner than the adjacent folded walls 131a, 131b and the cylindrical walls 134a, 134b or the wall can be formed from a less rigid material than that of the adjacent folded walls 131a, 131b and the cylindrical walls 134a, 134b.

If the walls 137, 138 in the molded body are not rigid enough, or do not span a sufficient width, to support the entire rocking seal 120 together with the walls that define the guidewire passageway 80, the walls 137, 138 can be radially extended at the seal proximal and distal ends as depicted in FIG. 13. Further, a rigid support member 136 can be provided around a periphery of the rocking seal 120 to provide further support if necessary. A vent 139 may be included in the support member 136 as necessary to allow external fluid to fill any space between the support member 136 and the rocking seal 120, and consequently enables the elongate neck 133 to constrict around the guidewire during guidewire advancement or other vacuum creating force.

The half rocking seal 125 is similar to the rocking seal 120 described above, except the cylindrical wall 134b, and folded wall 131b from the rocking seal are replaced with a large annular rib 135 that is continuously formed with the elongate neck 133 and extends radially to span the guidewire passageway 80. The half rocking seal 125 functions in the same manner as the rocking seal 120 to form a substantially fluid tight seal around the guidewire 14.

If the walls in the molded body are not rigid enough, or do not span a sufficient width, to support the entire rocking seal 120 or half rocking seal 125 together with the walls that define the guidewire passageway 80, the walls can be radially extended at the seal proximal and distal ends 137, 138 as depicted in FIG. 13, or the annular rib 135 can be extended further than as depicted in FIG. 14. Further, a rigid support member 136 can be provided around a periphery of either seal 120, 125 to provide further support if necessary. If the rigid support member 136 conforms or nearly conforms to the cylindrical wall periphery, a vent 139 may be included in the support member 136. The vent 139 allows external air to fill any space between the support member 136 and either seal 120, 125, and consequently enables the elongate neck 133 to constrict during guidewire advancement or other vacuum creating force.

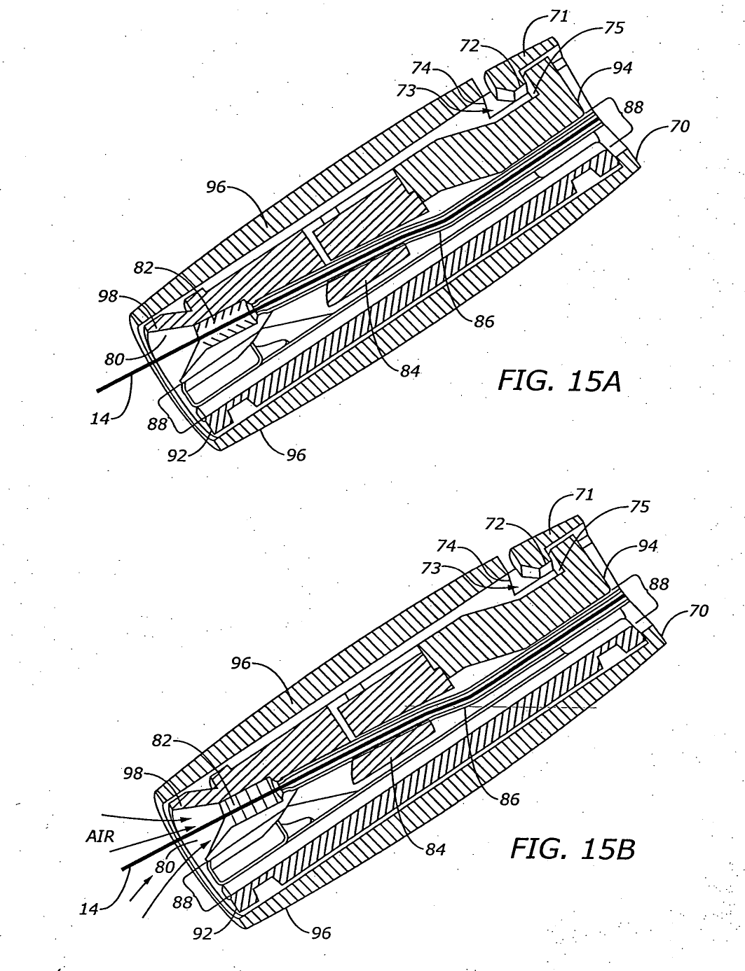

FIGS. 15A and 15B are sectional views of the guide member 10 with a modified passageway including active seals according to another embodiment of the invention. The guidewire 14 is illustrated extending through the guidewire passageway 80 including the entrance port 82 and tube 86. Fluid intake in a dye injection syringe, guidewire advancement through the tube 86, and other vacuum creating forces may draw air into the tube 86 by way of the entrance port 82. To prevent the inrushing air from reaching the catheter guidewire lumen 30, the entrance port is equipped with one or more annular flaps 85. Each flap 85 has a central aperture for slidingly receiving the guidewire 14. Each flap 85 is formed from a flexible material that is formed as a cone with a truncated tip that produces the central aperture. As depicted in FIG. 5A, the cone shaped flaps 85 are positioned with the smaller diameter portion proximal to the larger diameter portion, and the point where the flaps 85 are joined to the entrance port 82 forms an annular pocket that receives inrushing air when the guidewire is advanced through the guide member 10 and into the catheter guidewire lumen 30. As depicted in FIG. 15B, the flexible flaps 85 are configured to collapse from their biased conical shape, each collapsed flap forming fluid tight seals around the guidewire 14 as a result of the force from the inrushing fluid.

The flaps 85 are formed using a flexible polymer in an exemplary embodiment, although there are many materials that may be used to form the flaps 85 and provide a substantially fluid tight seal about the guidewire 14. Also, although the flaps 85 are shown to be positioned within the entrance port 82, they may be in any suitable position within the guidewire passageway 80, including the entrance port 82 and the tube 86.

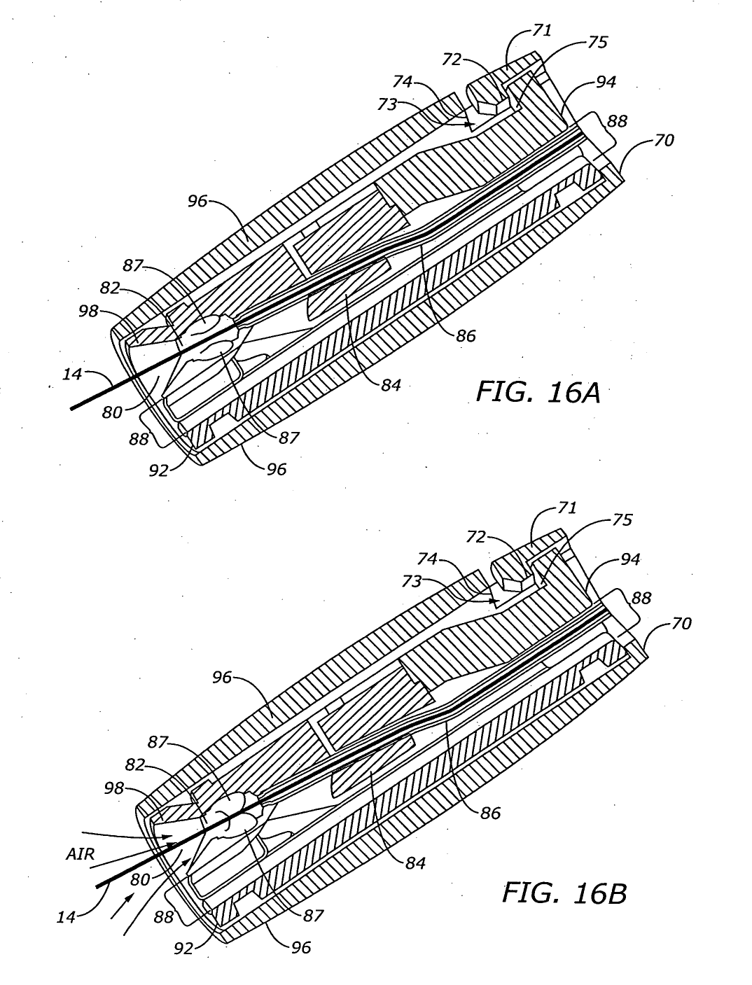

Another embodiment of the invention involving active seals is depicted in FIGS. 16A and 16B. The entrance port 82 includes at least one balloon 87 that is inflatable as a result of the force produced by inrushing fluid. As depicted in FIG. 16A, the balloon 87 does not substantially impinge on the entrance port 87 when deflated, and the deflated balloon 87 may even be positioned entirely outside of the guidewire passageway 80. As depicted in FIG. 16B, the inflated balloon 87 occludes the entrance port 82 and thereby prevent inrushing air from entering the tube 86.

The balloon 87 can be formed using any one of many known materials that may be used to form inflatable balloons as long as the inflated balloon 87 can provide a substantially airtight seal about the guidewire 14 without creating a detrimental amount of friction that would impede guidewire advancement. Also, although the balloon 87 is depicted to be positioned within the entrance port 82, the balloon 87 may be in any suitable position within the guidewire passageway 80.

The balloon 87 may have an annular shape that consequently surrounds the guidewire 14 by itself Alternatively, a plurality of balloons may be positioned around the guidewire 14 such that the balloons, when inflated, together form a single airtight seal. Any number of balloons may be used to provide the airtight seal about the guidewire 14.

Any of the above passive or active seals that are capable of being secured in the entrance port 82 or other guidewire passageway component may be secured using by a friction force. The various sealing bodies or assemblies may be made from, or housed in a material that has a high friction coefficient to prevent the bodies or assemblies from adjusting out of the desired position. The sealing bodies or assemblies may also be secured using an adhesive, a bracket or other fastening device.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing the exemplary embodiment or exemplary embodiments. It should be understood that various changes can be made in the function and arrangement of elements without departing from the scope of the invention as set forth in the appended claims and the legal equivalents thereof.

What is claimed is:

1. A catheter and guidewire exchange system, comprising:
a catheter, comprising:
an elongate shaft having an exterior surface, a proximal end, and a distal end,
a first lumen extending through the shaft from the shaft proximal end to the shaft distal end, and sized to receive a guidewire, and
a longitudinal guideway extending distally from the shaft proximal end, and enabling transverse access from the shaft exterior surface to the first lumen; and
a guide member, comprising:
a housing having a proximal end and a distal end,
a catheter passageway extending through the housing from the proximal end to the distal end and adapted to slidably receive the catheter,
a guidewire passageway extending from the housing proximal end into the catheter passageway, and comprising a tube adapted to merge the guidewire transversely through the guideway and into the first lumen, and
a fluid flow reduction body that is positioned in the guidewire passageway and impedes fluid flow therethrough.

2. The system according to claim 1, wherein the guidewire passageway further comprises a guidewire entrance port that is positioned proximal to the tube, and the fluid flow reduction body is secured within the guidewire entrance port.

3. The system according to claim 1, wherein the fluid flow reduction body comprises at least one annular flap having a central aperture for slidably receiving the guidewire.

4. The system according to claim 3, wherein the at least one annular flap is further shaped as a cone having first and second ends, the first end being truncated by the central aperture.

5. The system according to claim 4, wherein the first end is proximally disposed relative to the second end.

6. The system according to claim 3, wherein the annular flap is formed from a flexible material and is adapted to collapse and form a substantially airtight seal about the guidewire due to a force produced by inflowing air through the guidewire passageway.

7. The system according to claim 2, wherein the fluid flow reduction body comprises a plurality of the annular flaps.

8. The system according to claim 1, wherein the fluid flow reduction body is an integral part of the tube.

9. The system according to claim 8, wherein the fluid flow reduction body comprises a fixed reduced diameter tube region.

10. The system according to claim 9, wherein the fluid flow reduction body comprises an annular device secured to the tube and creating the fixed reduced diameter tube region.

11. The system according to claim 10, wherein the annular device surrounds the tube.

12. The system according to claim 10, wherein the annular device is secured to the tube interior.

13. The system according to claim 1, wherein the fluid flow reduction body comprises at least one balloon that is adapted to inflate and form a substantially airtight seal about the guidewire due to a force produced by inflowing air through the guidewire passageway.

14. The system according to claim 13, wherein the balloon has an annular shape.

15. The system according to claim 13, wherein the fluid flow reduction body comprises a plurality of the balloons that are adapted to inflate and together form the substantially airtight seal.

16. The system according to claim 13, wherein the balloon does not impinge on the guidewire passageway when the balloon is deflated.

17. The system according to claim 1, wherein the fluid flow reduction body comprises at least one o-ring body having a central opening for slidably receiving the guidewire.

18. The system according to claim 1, wherein the fluid flow reduction body comprises at least one disk having a slit for slidably receiving the guidewire.

19. The system according to claim 18, wherein the fluid flow reduction body comprises a plurality of the disks spaced apart from one another.

20. The system according to claim 19, wherein the slit in each disk is oriented at an angle with respect to a slit in an adjacent disk.

21. The system according to claim 20, wherein the slits are oriented at a 45° angle with respect to a slit in an adjacent disk.

22. The system according to claim 1, wherein the fluid flow reduction body comprises a capsule having openings for slidably receiving the guidewire, and a biocompatible gel composition contained in the capsule.

23. The system according to claim 22, wherein the biocompatible gel composition comprises silicone.

24. The system according to claim 1, wherein the fluid flow reduction body comprises an annular body including a central o-ring body with an inner diameter, and a first conical flexible body formed continuous with the o-ring body and having a central aperture for slidably receiving the guidewire.

25. The system according to claim 24, wherein the first conical flexible body is proximally disposed relative to the o-ring body.

26. The system according to claim 24, wherein the annular body further includes a second conical body formed continuous with the o-ring body and having a central aperture adapted to slidably receive the guidewire, the second conical body being disposed opposite the first conical body with respect to the o-ring body.

27. The system according to claim 1, wherein the fluid flow reduction body comprises a solid body having an hour glass shaped passageway extending therethrough, the passageway including a neck region adapted to slidably receive the guidewire, the neck region having a smaller diameter than the rest of the passageway.

28. The system according to claim 27, wherein the solid body comprises a continuous wall that defines the passageway, the continuous wall comprising a flexible material that surrounds at least the neck region.

29. The system according to claim 28, wherein the continuous wall further comprises regions adjacent to the flexible material, the adjacent regions being substantially more rigid than the flexible material.

30. The system according to claim 29, wherein the flexible material surrounding the neck region is thinner than the adjacent regions.

31. The system according to claim 1, wherein the fluid flow reduction body comprises:
a substantially cylindrical wall;
an inwardly folding wall continuously formed with the substantially cylindrical wall and defining an annular pocket; and
a wall defining an elongate neck continuously formed with the inwardly folding wall and adapted to slidingly receive the guidewire.

32. The system according to claim 31, wherein the wall defining the elongate neck is formed from a flexible material.

33. The system according to claim 32, wherein the wall defining the elongate neck is more flexible than the substantially cylindrical wall and the inwardly folding wall.

34. The system according to claim 32, wherein the wall defining the elongate neck is thinner than the substantially cylindrical wall and the inwardly folding wall.

35. An apparatus for advancing and retracting a guidewire and a catheter having a lumen, an exterior surface, and a longitudinal guideway that enables transverse access from the catheter exterior surface to the lumen, the apparatus comprising:
a housing having a proximal end and a distal end,
a catheter passageway extending through the housing from the proximal end to the distal end and adapted to slidably receive the catheter,
a guidewire passageway extending from the housing proximal end into the catheter passageway, and comprising a tube adapted to merge the guidewire transversely through the guideway and into the first lumen, and
a fluid flow reduction body that is positioned in the guidewire passageway and impedes fluid flow therethrough.

36. The apparatus according to claim 35, wherein the guidewire passageway further comprises a guidewire entrance port that is positioned proximal to the tube, and the fluid flow reduction body is secured within the guidewire entrance port.

37. The apparatus according to claim 35, wherein the fluid flow reduction body comprises at least one annular flap having a central aperture for slidably receiving the guidewire.

38. The apparatus according to claim 37, wherein the at least one annular flap is further shaped as a cone having first and second ends, the first end being truncated by the central aperture.

39. The apparatus according to claim 38, wherein the first end is proximally disposed relative to the second end.

40. The apparatus according to claim 37, wherein the annular flap is formed from a flexible material and is adapted to collapse and form a substantially airtight seal about the guidewire due to a force produced by inflowing air through the guidewire passageway.

41. The apparatus according to claim 37, wherein the fluid flow reduction body comprises a plurality of the annular flaps.

42. The apparatus according to claim 35, wherein the fluid flow reduction body is an integral part of the tube.

43. The apparatus according to claim 42, wherein the fluid flow reduction body comprises a fixed reduced diameter tube region.

44. The apparatus according to claim 43, wherein the fluid flow reduction body comprises an annular device secured to the tube and creating the fixed reduced diameter tube region.

45. The apparatus according to claim 44, wherein the annular device surrounds the tube.

46. The apparatus according to claim 44, wherein the annular device is secured to the tube interior.

47. The apparatus according to claim 35, wherein the fluid flow reduction body comprises at least one balloon that is adapted to inflate and form a substantially airtight seal about the guidewire due to a force produced by inflowing air through the guidewire passageway.

48. The apparatus according to claim 47, wherein the balloon has an annular shape.

49. The apparatus according to claim 47, wherein the fluid flow reduction body comprises a plurality of the balloons that are adapted to inflate and together form the substantially airtight seal.

50. The system according to claim 47, wherein the balloon does not impinge on the guidewire passageway when the balloon is deflated.

51. The apparatus according to claim 35, wherein the fluid flow reduction body comprises at least one o-ring body having a central opening for slidably receiving the guidewire.

52. The apparatus according to claim 35, wherein the fluid flow reduction body comprises at least one disk having a slit for slidably receiving the guidewire.

53. The apparatus according to claim 52, wherein the fluid flow reduction body comprises a plurality of the disks spaced apart from one another.

54. The apparatus according to claim 53, wherein the slit in each disk is oriented at an angle with respect to a slit in an adjacent disk.

55. The apparatus according to claim 54, wherein the slits are oriented at a 45° angle with respect to a slit in an adjacent disk.

56. The apparatus according to claim 35, wherein the fluid flow reduction body comprises a capsule having openings for slidably receiving the guidewire, and a biocompatible gel composition contained in the capsule.

57. The apparatus according to claim 56, wherein the biocompatible gel composition comprises silicone.

58. The apparatus according to claim 35, wherein the fluid flow reduction body comprises an annular body including a central o-ring body with an inner diameter, and a first conical flexible body formed continuous with the o-ring body and having a central aperture for slidably receiving the guidewire.

59. The apparatus according to claim 58, wherein the first conical flexible body is proximally disposed relative to the o-ring body.

60. The apparatus according to claim 58, wherein the annular body further includes a second conical body formed continuous with the o-ring body and having a central aperture adapted to slidably receive the guidewire, the second conical body being disposed opposite the first conical body with respect to the o-ring body.

61. The apparatus according to claim 35, wherein the fluid flow reduction body comprises a solid body having an hour glass shaped passageway extending therethrough, the passageway including a neck region adapted to slidably receive the guidewire, the neck region having a smaller diameter than the rest of the passageway.

62. The apparatus according to claim 61, wherein the solid body comprises a continuous wall that defines the passageway, the continuous wall comprising a flexible material that surrounds at least the neck region.

63. The apparatus according to claim 62, wherein the continuous wall further comprises regions adjacent to the flexible material, the adjacent regions being substantially more rigid than the flexible material.

64. The apparatus according to claim 63, wherein the flexible material surrounding the neck region is thinner than the adjacent regions.

65. The apparatus according to claim 35, wherein the fluid flow reduction body comprises:
a substantially cylindrical wall;
an inwardly folding wall continuously formed with the substantially cylindrical wall and defining an annular pocket; and
a wall defining an elongate neck continuously formed with the inwardly folding wall and adapted to slidingly receive the guidewire.

67. The apparatus according to claim 66, wherein the wall defining the elongate neck is formed from a flexible material.

68. The apparatus according to claim 67, wherein the wall defining the elongate neck is more flexible than the substantially cylindrical wall and the inwardly folding wall.

69. The apparatus according to claim 67, wherein the wall defining the elongate neck is thinner than the substantially cylindrical wall and the inwardly folding wall.
